(19) United States
(12) Patent Application Publication
(10) Pub. No.: US 2023/0063919 A1
(43) Pub. Date: Mar. 2, 2023

(54) SCENTING DEVICE FOR A VEHICLE

(71) Applicant: Supair-Tel AG, Glattbrugg (CH)

(72) Inventor: Rudolf Heinrich GUGGENHEIM, Wollerau (CH)

(21) Appl. No.: 17/900,415

(22) Filed: Aug. 31, 2022

(30) Foreign Application Priority Data
Sep. 1, 2021 (EP) 21194248.7

(51) Int. Cl. B60H 3/00

(52) U.S. Cl. CPC B60H 3/0028

(57) ABSTRACT

A scenting device is provided for scenting an interior of a vehicle. The scenting device comprises a housing and a scent container arranged in the housing. The housing comprises a holding device for attaching the scent container.

### FIELD OF THE INVENTION

The invention relates to a scenting device for a vehicle with a housing and a scent container arranged in the housing. The housing comprises a holding device for attaching the scenting device to a ventilation in the interior of a vehicle. Furthermore, the housing comprises an inlet side and an outlet side. On the inlet side, an inlet opening is arranged for air to flow into the housing and on the outlet side, an outlet opening is arranged for air to flow out of the housing. The outlet side has an elongated shape with a long side and a short side.

### BACKGROUND

Scenting devices are known which can be suspended in the interior of a vehicle and have, for example, a material impregnated with a scent. Such devices can be manufactured at low cost, but their scenting effect can be uneven and the scenting body, which is usually suspended from the interior rear-view mirror, can disturb visibility.

Furthermore, containers with scent are known which are attached to a ventilation grille in the interior of a vehicle. The container for the scent has inlet openings so that air from the ventilation system flows into the container, comes into contact with the scent and, enriched with scent molecules, leaves the container through outlet openings. Such containers can be attached to and removed from the ventilation grille by means of a clamping part.

### SUMMARY

The object is to provide a scenting device which is easy to operate.

This object is solved by a scenting device with the features of claim 1.

The scenting device according to the invention comprises a housing in which a scent container is arranged. The housing comprises
a holding device for attaching the scenting device to a ventilation in the interior of a vehicle. The term “vehicle” includes land vehicles, such as passenger cars and trucks, and also aircraft.
an inlet side at which an inlet opening is arranged for the inflow of air into the housing. If the air scenting device is attached to the ventilation of the vehicle, the inlet side is in particular directed towards the ventilation.
an outlet side, in particular opposite the inlet side, on which an outlet opening for the outflow of air from the housing is arranged. If the scenting device is attached to the ventilation of the vehicle, the outlet side faces away from the ventilation in particular.

In particular, the outlet side has an elongated shape with a long side and a short side. For example, the outlet side may have a rectangular shape. The rectangular shape has a long side and a short side. For example, if the rectangle is a square, the long side and short side are of equal length. That is, in particular, the long side and short side may be of different lengths or of the same length.

The scenting device comprises a sliding device with a sliding container arranged in the housing. The scent container is arranged in the sliding container. By means of the sliding device, the outlet and/or inlet opening can be at least partially, in particular completely, closed by means of a sliding movement. The scenting device can thus be opened and closed in a simple manner. It is therefore very easy to operate.

The sliding device is in particular adapted such that the scent container moves relative to the housing during the sliding movement. This has the advantage that the sliding movement can be carried out without errors. There is no relative movement between the scent container and the sliding device during which a snagging or other blockage could occur.

In particular, the scenting container is arranged in the sliding container such that the scent container does not rub along the housing during the sliding movement. Thus, no blockages can occur between the scent container and the housing during the movement. The sliding movement is controlled within the housing.

Advantageously, the housing comprises webs on which the sliding container is supported or which the sliding container touches. This creates a relative movement between the webs and the sliding container. Due to the small contact surface between the bar and the sliding container, friction is reduced and the sliding container can be moved in a controlled manner within the housing.

In particular, the sliding device has a handle, in particular a plate-shaped handle. By actuating the handle, the sliding device can be moved, whereby the handle does not protrude through either the outlet side or the inlet side. This allows the scenting device to have a compact geometry, because the inlet side and outlet side are as completely available as possible for inlet and outlet openings.

In particular, the outlet opening is designed in the form of an elongated slot. In particular, several slots can be arranged next to each other on the outlet side. The elongated slot is arranged obliquely to the longitudinal side. I.e. the slot does not run parallel to the longitudinal side. In particular, the elongated slot is at an angle of at least 10°, in particular at least 20°, in particular at least 30°, to the longitudinal side.

Furthermore, the elongated slot can also be arranged at an angle to the short side. In particular at an angle of at least 10°, in particular at least 20°, in particular at least 30°, to the short side. This means that the elongated slot is neither parallel to the long side nor parallel to the short side. If the short side and the long side are at an angle of 90° to each other, the elongated slot can be arranged at an angle of 45° to the short side and at an angle of 45° to the long side, for example.

If the elongated slots were arranged parallel to the short side, the slots could extend at most over the length of the short side. The slots would be relatively short and the more slots would have to be arranged on the outlet side so that a certain volume of air could flow through the container. Since a minimum distance is required between two slots for design reasons, the outlet side would have to be dimensioned accordingly. If, on the other hand, the slots are arranged at an angle to the short side, the problem described can be avoided. The slots can be dimensioned with a greater length.

If the elongated slots were arranged parallel to the long side, a sliding device with which the slots can be closed and opened or covered and uncovered would be structurally difficult to manage. A sliding movement in the direction of the short side is constructively difficult and a sliding movement in the direction of the long side would require an interruption of the slots.

For design reasons, it is therefore advantageous to arrange the slot-shaped outlet openings obliquely to the long side and/or to the short side.

Advantageously, the scenting device comprises three elongated slots, in particular wherein the three elongated slots are arranged on the outlet side. The scenting device may also have further slots.

Advantageously, the three elongated slots are parallel to each other and/or they are arranged equidistant to each other. Advantageously, the three elongated slot are of equal length. In particular, the elongated slots extend substantially over the entire short side of the outlet side. Such an arrangement of the elongated slots has the advantage that they can be arranged as compactly as possible on the outlet side.

In particular, the scent container of the scenting device is removable. It may be in the form of a stick. The housing can have a housing opening through which the scent container can be inserted into and removed from the housing. A removable scent container has the advantage that it can be replaced after use, but the housing of the scent device can continue to be used.

In particular, the housing has a housing cover with which the housing opening can be opened and closed. The housing cover is attached to the housing by means of a hinge. The hinge allows the enclosure lid to be opened and closed in a simple manner by means of a rotary movement. The housing cover remains attached to the housing and cannot get lost during the replacement of the scent container.

Advantageously, there is only one opening in the housing. Only through this opening can the scent container be inserted into the housing. When the housing cover is closed, the housing cover can in particular be locked in place so that the housing cover does not open unintentionally during use.

Advantageously, the housing cover extends along a section of the longitudinal side. This has the advantage that the housing opening is relatively large and the scent container can be conveniently inserted into the housing.

Furthermore, the scenting device can be adapted such that the housing cover can only be closed when the scent container is inserted in the housing in the correct position and/or correctly aligned, wherein there is only exactly one correct position and/or only exactly one correct alignment. In particular, the scent container has a recess for this purpose, in which a protrusion of the housing or of the sliding device engages. If the recess and the protrusion do not engage with each other, the scent container cannot be fully inserted into the housing and the housing lid cannot be closed. The user notices the misplacement and corrects it.

It should be noted that in a scenting device, the described inclined arrangement of the inlet and/or outlet openings can also be provided independently of the embodiment with sliding device claimed in claim 1. For example, a scenting device for a vehicle may be provided with a housing and a scent container arranged in the housing, wherein the housing comprises
a holding device for attaching the scenting device to a ventilation in the interior of a vehicle,
an inlet side, on which an inlet opening for the inflow of air into the housing is arranged,
an outlet side, in particular opposite the inlet side, on which an outlet opening for the outflow of air from the housing is arranged,

wherein the outlet opening (5) is an elongated slot and the elongated slot is arranged obliquely to the longitudinal side (61) of the outlet side (6), in particular is also arranged obliquely to the short side (62) of the outlet side (6).

In particular, the elongated slot is arranged at an angle of at least 10°, in particular at least 20°, in particular at least 30°, to the longitudinal side (61) of the outlet side (6) and/or to the short side (61) of the outlet side (6).

In particular, the scenting device has at least three elongated slots.

### BRIEF DESCRIPTION OF THE DRAWINGS

Further embodiments, advantages and applications of the invention will be apparent from the dependent claims and from the following description based on the figures. The figures show:

FIG. 1 a scenting device with a view of the front and a scent container which has not been inserted;

FIG. 2 the scenting device with a view of the rear side, whereby two rubber caps are coupled to the scenting device;

FIG. 3 the rear view of the scenting device with a spreader element coupled to the scenting device;

FIG. 4 an exploded view of the scenting device without the scent container; and

FIGS. 5a to 5f a rubber cap in different views.

### DETAILED DESCRIPTION OF THE INVENTION

### Overview

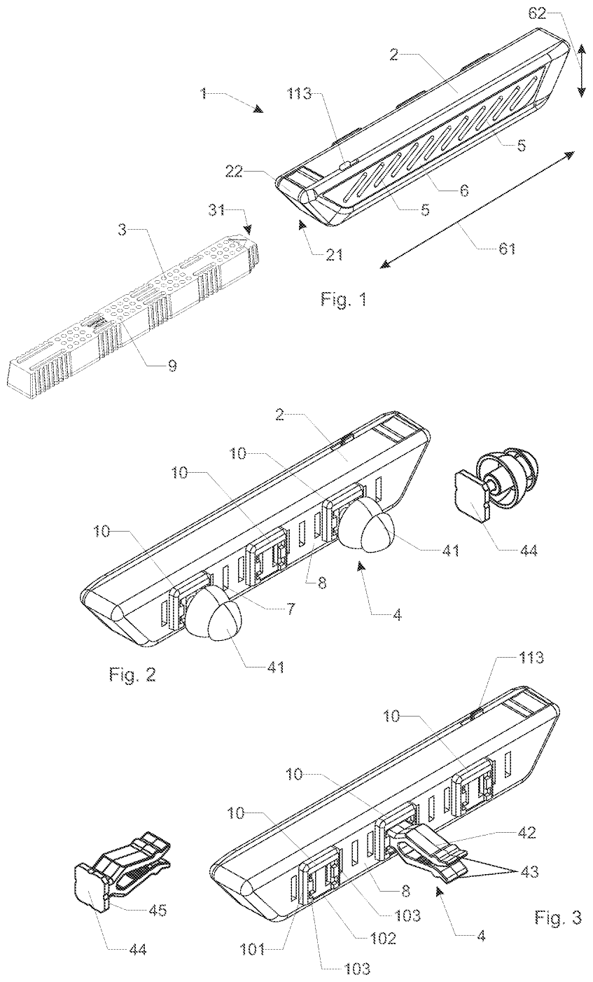

FIGS. 1 to 4 show a scenting device 1 according to the invention for a vehicle with a housing 2 and a scent container 3 arranged in the housing 2. The scent container 3 is replaceable. It can be removed from the housing 2 and a new scent container 3 can be inserted into the housing 2. The scenting device 1 has holding devices 4 with which the scenting device 1 can be mounted on a ventilation system in the interior of the vehicle.

FIG. 1 shows the front side of the scenting device 1. Outlet openings 5 are arranged at the front of the scenting device 1. The front side is therefore also referred to as the outlet side 6. In addition, outlet openings may also be provided on one or both side walls of the housing 2 and/or on its top side and/or on its bottom side.

FIGS. 2 and 3 show the scenting device 1 as seen from the rear. Inlet openings 7 are arranged at the rear of the scenting device 1. The rear side is therefore also referred to as the inlet side 8.

If the scenting device 1 is mounted on the ventilation system in the passenger compartment of the vehicle, the inlet side 8 is directed towards the ventilation system and the outlet side 6 is directed away from the ventilation system. Air flows out of the ventilation through the inlet openings 7, into the housing 2 and into the scent container 3 arranged in the housing 2. The air comes into contact with the scent in the scent container 3, is enriched with scent molecules and leaves the housing 2 of the scenting device 1 through the outlet openings 5. The air reaches the interior of the vehicle and scents the interior.

Scent Container

The scent container 3, as shown in FIG. 2, is provided as a replaceable scent stick, wherein the scent stick comprises as material a composite material of at least one polymer, in particular an elastomer, and the scent, or consists of such a material. In this case, the polymer is enriched with the scent and acts as its carrier. Preferably, the scent stick has a plurality of passage openings 9 through which the air emerging from the ventilation slot can flow. By providing the passage openings 9, the surface area is increased at which the air exiting the ventilation slot comes into contact with the scent, which results in an increased enrichment of the air flowing through the scenting device 1 with scent molecules. The passage openings 9 are preferably aligned with the direction of flow of the air from the ventilation slot. Alternatively, the scent may be provided in the form of granules contained in a cage.

As shown in FIG. 1, the housing 2 has a housing opening 21 at one of its ends, which can be closed with a housing cover 22. To replace the scent container 3, the user opens the housing cover 22, which is attached to the housing by a rotary hinge and thus remains connected to the housing 2 even in the open position.

If the user wants to insert the new scent container 3, he must insert it in the correct direction into the housing 2 and into a sliding container 11. This is because a recess 31 is provided at the front end of the scent container 3, in which a protrusion of the housing 2 or of the sliding container 11 engages in the inserted position. If the recess 31 and the protrusion do not engage, the scent container 3 cannot be fully inserted into the housing 2 and the housing cover 22 cannot be closed. Once the user has correctly inserted the scent container 3 into the housing 2, he can close the housing cover 22. When closing, the housing cover 22 locks into place on the housing 2 so that the housing cover 22 does not open unintentionally.

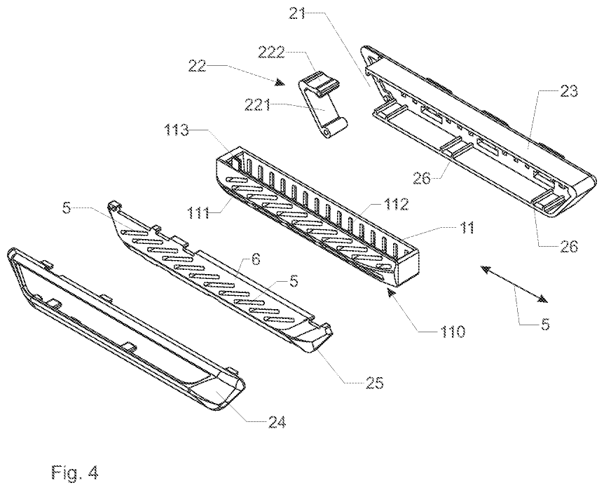

It is clearly visible in FIG. 4 that the housing cover 22 has a curved shape. The longer section 221 of the housing cover 22 forms the lateral end of the housing 2. The shorter section 222 of the housing cover 22 extends along a section of the longitudinal side of the housing 2. The curved shape of the housing cover 22 has the advantage that the housing opening 21 is correspondingly large and the scent container 3 can be conveniently inserted.

Design of the Outlet Side

As shown in FIG. 1, the housing 2 and thus also the outlet side 6 has an elongated shape with a long side 61 and a short side 62. Several outlet openings 5 are arranged on the outlet side 6. They each have the shape of an elongated slot and are arranged parallel and equidistant to each other.

The outlet openings 5 are each arranged obliquely to the long side 61 and obliquely to the short side 62. In other words, the outlet openings 5 are arranged neither parallel to the longitudinal side 61, nor parallel to the short side 62. In the present embodiment, oblique means that the outlet openings 5 designed as slots are arranged at an angle of 45° to the long side 61 and at an angle of 45° to the short side 62. The outlet openings 5 each extend approximately over the entire length of the short side 62.

Holding Devices

Three coupling devices 10 are arranged on the inlet side 8. A holding device 4 can be coupled to each of the three coupling devices 10. The three coupling devices 10 are spaced apart and inlet openings 7 are arranged between them.

FIG. 2 shows a first variant for mounting the air freshener 1 on the vehicle's ventilation system. A holding device 4 with a rubber cap 41 is coupled to each of the outer coupling devices 10. The middle coupling device 10 is left free and is not coupled to any holding device 4.

The rubber caps 41 are made of a resilient material and are configured to be clamped between two slats of the ventilation in the vehicle. The holding devices 4 with the rubber caps 41 can thus be inserted in a holding manner into a ventilation slot in the vehicle interior. In other words, the dimensions of the rubber caps 41 are determined in dependence on the dimensions, in particular the width, of the ventilation slot in such a way that the air freshening device 1 can be attached to the ventilation slot by inserting/inserting the rubber caps 41 into the ventilation slot where they are held in a clamping manner by the latter. The rubber caps 41 are attachable to the holding devices 4. This allows the rubber caps 41 to be replaced in a simple manner.

The housing 2 is preferably elongated. If it is mounted on a ventilation slot, it extends along the ventilation slot. Both holding devices 4 are arranged in the same ventilation slot. Of course, it is also possible to arrange the housing 2 transversely to the ventilation slots, in which case the holding devices 4 are placed in different ventilation slots.

FIG. 3 shows a second variant for mounting the air freshener 1 on the vehicle's ventilation system. The outer coupling devices 10 are left free and are not coupled to any holding device 4. Only on the middle coupling device 10 is a holding device 4 arranged, which has a spreader element 42. The spreader element 42 comprises two spreader members 43 and can be clamped between two slats of the vehicle's ventilation like the rubber caps 41.

The holding device 4 with the rubber cap 41 and the holding device 4 with the spreader element 42 represent different types of holding devices 4, which are structurally different.

All three coupling devices 10 are identically designed. That is, both the holding devices 4 with the rubber caps 41 and the holding device 4 with the spreader element 42 can be coupled to all three coupling devices and thus be interchangeable with each other. According to the claims, each of the holding devices 4 can thus be coupled to one of the at least one coupling device 10.

As shown in FIGS. 2 and 3, the coupling devices 10 couple to the holding devices 4 in the form of a snap-fit. The coupling devices 10 comprise a square frame 101. The frame 101 is closed on three sides and has a frame opening 102 on its underside. The frame 101 also has two gaps 103 arranged on each of the left and right sides.

The holding device 4 comprises a flat, square plate 44 to which either the rubber caps 41 or the spreader element 42 are connected, depending on the type of holding device 4. Protrusions 45 are arranged on the plate 44, which can engage in the gaps 103 of the frame 101 of the coupling device 10 in a snapped-in position.

A user can couple the holding device 4 to the coupling device 10 by pushing the plate 44 of the holding device 4 from the underside through the frame opening 102 below the frame 101. In doing so, the frame 101 deforms elastically because the protrusions 45 press on the frame 101. The user pushes the plate 44 further until the protrusions 45 appear in the gaps 103. The frame 101 deforms back and the plate 44 is snapped into the frame 101. Holding device 4 and coupling device 10 are coupled together.

If a user wishes to uncouple the holding device 4 from the coupling device 10, the user moves the holding device 4 downwards so that the plate 44 is guided out of the frame 101 through the frame opening 102. The user must apply a certain amount of force for this movement, i.e. for the disengagement of the snap-fit, as the frame 101 again deforms elastically due to the protrusions 45.

As already described, different variants are available to the user for mounting the air freshener 1 to the ventilation in the vehicle. On the one hand, the user can use the holding devices 4 with the rubber caps 41 for mounting, on the other hand, the holding device 4 with the spreader element 42. The user thus has a kit which comprises, on the one hand, the housing 2 with the scent container 3 and, on the other hand, the different types of holding devices 4. In addition, the kit may also include rubber caps 41 of different sizes. I.e. in this case the user has a total of three variants at his disposal for mounting the scenting device 1 on the ventilation in the interior of the vehicle. The mounting is carried out by means of the smaller rubber caps 41, by means of the larger rubber caps 41 or by means of the spreader element 42. The user chooses from the three variants the one which is best suited for mounting the scenting device 1 depending on the design of the ventilation.

Sliding Device

FIG. 4 shows an exploded view of the individual parts of the scenting device 1. The scent container 3 is not visible. The housing 2 comprises a rear housing cover 23, a front housing frame 24 which frames a front housing cover 25. The housing cover 25 includes the aforementioned outlet openings 5 on the outlet side 6. Also shown is the aforementioned housing cover 22, which can close the housing opening 21.

A sliding device with a sliding container 11 is arranged inside the housing 2. The sliding container comprises a bottom 110, a front wall with inclined outlet openings 111 and a rear wall with vertical inlet openings 112. There is space between the walls of the sliding container 11 so that the scent container 3 can be arranged inside the sliding container 11.

On the inside of the housing 2, there are webs 26 on which the sliding container 11 rests on the bottom or which it touches with its side walls. This keeps the touching area between the housing 2 and the sliding container 11 as small as possible. The sliding container 11 can be easily moved within the housing 2.

To move the sliding container 11, a plate-shaped handle 113 is arranged on its upper side, which protrudes from the housing 2 through the upper side of the housing 2. The handle 113 thus does not protrude through either the inlet side 8 or the outlet side 6. The handle 113 is also visible in FIG. 1. By actuating the handle 113, the user can move the sliding container 11 longitudinally relative to the housing 2. The sliding container 11 is thereby moved back and forth in the direction of the double arrow 114 shown in FIG. 4.

Since the scent container 3 is arranged within the sliding container 11 and does not touch the housing 2, the scent container 3 moves in the same manner as the sliding container 11 relative to the housing 2. As a result, the scent container 3 does not rub against the housing 2 during the movement. Friction only occurs between the sliding container 11 and the webs 26 of the housing 2. The movement thus occurs in a controlled manner, with only slight friction, and is independent of whether the scent container 3 has been correctly inserted into the sliding container 11 by the user.

By means of the movement of the sliding container 11, the user can open and close the inlet openings 7 and the outlet openings 5. In the closed state, the struts of the sliding container 11 cover the inlet openings 7 and the outlet openings 5. Air cannot flow through the scenting device 1. In the open state, the outlet openings 111 of the sliding container 11 are congruent with the outlet openings 5 of the housing 2 and the inlet openings 112 of the sliding container 11 are congruent with the inlet openings 7 of the housing 2. Air can flow through the scenting device 1 and be enriched with scent molecules of the scent container 3.

Design of the Rubber Caps

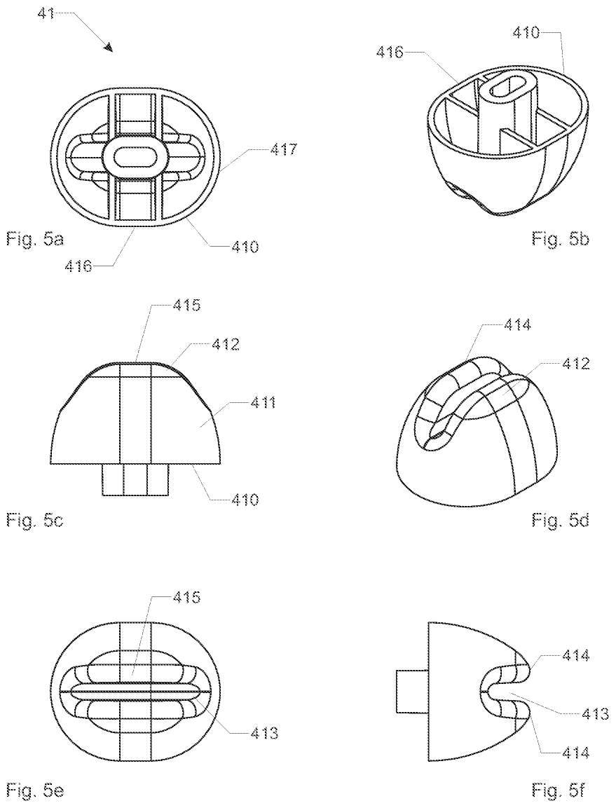

FIGS. 5a to 5f show the rubber caps 41 arranged on the holding devices 4 in detailed view and from different perspectives. The rubber cap 41 has a base surface 410, an outer surface 411 and an upper surface 412 opposite the base surface 410. Since the rubber cap 41 shown in FIG. 6 has a rounded upper surface 412, the upper surface 412 forms part of the outer surface 411.

The base surface is the surface of the rubber cap which, in the assembled state, faces the housing of the scenting device. The upper surface is the surface of the rubber cap which, in the assembled state, is directed away from the housing of the scenting device.

An elongated groove 413 is arranged on the upper surface 412 of the rubber cap 41. The groove 413 separates the upper surface 412 into two parallel upper edges 414, each of these upper edges 414 having a rectilinear section 415.

The base 410 of the rubber cap 41 has a perimeter or an edge line with two rectilinear sections 416. These two rectilinear sections 416 are joined by two semicircles 417. The base 410 thus resembles an elliptical shape, although it is precisely not an elliptical shape because the rim line has rectilinear sections 416.

While preferred embodiments of the invention are described in the present application, it should be clearly noted that the invention is not limited to these and may also be carried out in other ways within the scope of the following claims.

1. A scenting device for a vehicle, the scenting device comprising:
a housing; and
a scent container arranged in the housing,
wherein the housing comprises
a holding device for attaching the scenting device to a ventilation system in the interior of a vehicle,
an inlet side at which an inlet opening is arranged for the inflow of air into the housing,
an outlet side, in particular opposite the inlet side, on which an outlet opening for the outflow of air from the housing is arranged,
wherein the scenting device has a sliding device with a sliding container arranged in the housing, the scent container being arranged in the sliding container, and
wherein the outlet opening and/or the inlet opening can be at least partially closed by a sliding movement sliding device.

2. The scenting device according to claim 1, wherein the sliding device is adapted such that the scent container is displaced relative to the housing during the sliding movement.

3. The scenting device according to claim 1, wherein the scent container is arranged in the sliding container such that the scent container does not rub along the housing during the sliding movement.

4. The scenting device according to claim 1, wherein the housing has webs on which the sliding container is supported.

5. The scenting device according to claim 1, wherein the sliding device has a handle, in particular a plate-shaped handle, by the actuation of which the sliding device is displaceable, wherein the handle protrudes neither through the outlet side nor through the inlet side.

6. The scenting device according to claim 1, wherein the scent container has a recess into which a protrusion of the sliding container protrudes when the scent container is inserted.

7. The scenting device according to claim 1, wherein the outlet side has an elongate shape with a longitudinal side and a short side, and wherein the outlet opening is an elongate slot and the elongate slot is arranged obliquely to the longitudinal side of the outlet side, in particular is also arranged obliquely to the short side of the outlet side.

8. The scenting device according to claim 7, wherein the elongated slot is arranged at an angle of at least 10°, in particular at least 20°, in particular at least 30°, to the longitudinal side of the outlet side and/or to the short side of the outlet side.

9. The scenting device according to claim 7, comprising at least three elongated slots.

10. The scenting device according to claim 9, wherein the elongated slots are arranged parallel to each other, and/or equidistant to each other, and/or of equal length.

11. The scenting device according to claim 7, wherein the elongated slot extends over substantially the entire short side of the outlet side.

12. The scenting device according to claim 1, wherein the scent container can be inserted into the housing through a housing opening and removed therefrom again, in particular wherein the scent container is in the form of a scent stick.

13. The scenting device according to claim 12, wherein the housing opening can be opened and closed by means of a housing cover, wherein the housing cover is arranged on the housing by means of a rotary hinge,
in particular wherein exactly one single housing opening is present,
in particular wherein the housing cover is latched in the closed state.

14. The scenting device according to claim 13, wherein a part of the housing cover extends along the longitudinal side.

15. The scenting device according to claim 13, wherein the housing cover can only be closed when the scent container is inserted in the housing in the correct position and/or correctly aligned, wherein there is only exactly one correct position and/or only exactly one correct alignment.
